(19) United States
(12) Patent Application Publication
(10) Pub. No.: US 2011/0112652 A1
(43) Pub. Date: May 12, 2011

(54) JOINT PROSTHESIS

(76) Inventors: Henrik Hansson, Vreta Kloster (SE); Lars Oster, Lidkoping (SE); Etzner Mikael, Varberg (SE)

(21) Appl. No.: 12/989,175

(22) PCT Filed: May 5, 2009
(86) PCT No.: PCT/SE09/50487

§ 371 Date: Dec. 15, 2010

(30) Foreign Application Priority Data
May 6, 2008 (SE) 0801001-9

(51) Int. Cl. A61F 2/42

(52) U.S. Cl. 623/21.16; 623/21.15

(57) ABSTRACT

The present invention relates to a joint prosthesis (1) comprising two prosthesis components (2, 3) intended to be applied respectively to the trapezium (38) and the first metacarpal bone (39) of a thumb joint, each prosthesis component having a first or second screw-like part (4, 5) intended to be screwed firmly or be fastened to one of said bones (38 or 39) by means of bone cement, the one prosthesis component (2) having an articulating socket element (6) and the other prosthesis component (3) an articulating ball element (7). The fact that at least the prosthesis component (2) with the socket element (6) has an abutment portion (20) for abutment against the bone to which the screw-like part (4) of the prosthesis component is intended to be firmly screwed or be fastened by means of bone cement, normally the trapezium (38), prevents this prosthesis component from sinking into said bone.

The present invention relates to a joint prosthesis comprising two prosthesis components intended to be applied respectively to the trapezium and the first metacarpal bone of a thumb joint. Each prosthesis component has a first or second screw-like part intended to be fastened, e.g. screwed firmly, to one of said bones or alternatively be fastened by means of bone cement. One prosthesis component has an articulating socket element and the other prosthesis component an articulating ball element.

There are various concepts for applying a joint prosthesis to a thumb joint, both with and without bone cement. Application without bone cement often entails problems of the prosthesis becoming loose. This applies particularly on the trapezium side. There is also risk that the prosthesis component on the trapezium side may “settle in”, i.e. sink into the ulna and assume a quite different angle, with consequent subluxation of the prosthesis.

The object of the present invention is therefore to eliminate the problem of prosthesis components loosening or “settling in”. This is achieved by the prosthesis having the characteristics indicated by the accompanying claims 1 and 6 respectively.

The fact that at least the prosthesis component with the socket element has an abutment portion for abutment against the bone to which the screw-like part of the prosthesis component is intended to be firmly screwed or be fastened by means of bone cement, i.e. normally the trapezium, prevents this prosthesis component from sinking into that bone.

As the prosthesis component with the socket element is for example intended to be screwed firmly into the trapezium with the limited space available in this bone, the screw-like part of this prosthesis component has according to the invention a length substantially corresponding to the length of the trapezium minus about 2.5 mm. The result is a contact surface of optimum size between the screw-like part and the bone, and optimum stability of primary fixation of this prosthesis component.

The above and other features characterising the invention are explained below in more detail with reference to the attached drawings, in which

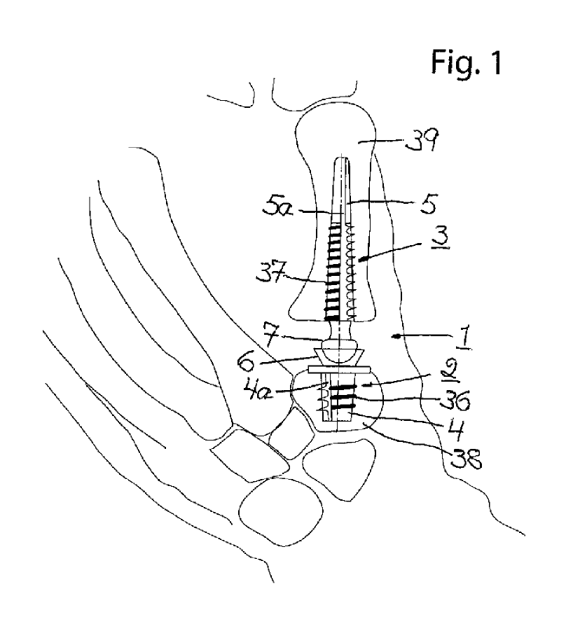

FIG. 1 depicts in schematic side view a thumb joint prosthesis according to the invention, implanted in a hand;

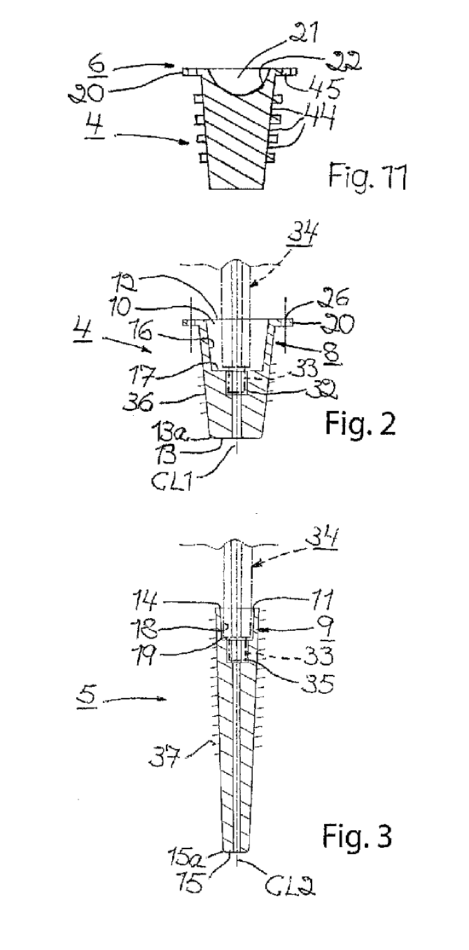

FIG. 2 depicts in schematic section a first screw-like part which forms part of the thumb joint prosthesis according to FIG. 1;

FIG. 3 depicts in schematic section a second screw-like part which forms part of the thumb joint prosthesis according to FIG. 1;

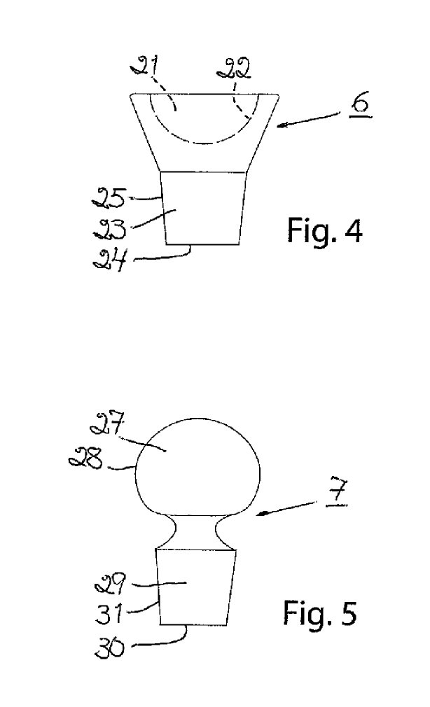

FIG. 4 depicts in schematic side view an articulating socket element which forms part of the thumb joint prosthesis according to FIG. 1;

FIG. 5 depicts in schematic side view an articulating ball element which forms part of the thumb joint prosthesis according to FIG. 1;

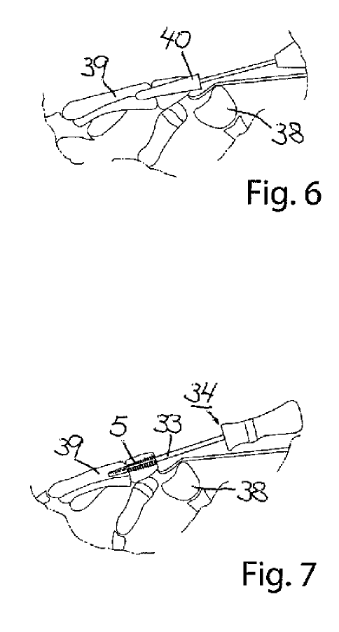

FIGS. 6-10 depict schematically various stages in applying the screw-like parts which form part of the thumb joint prosthesis according to FIG. 1; and

FIG. 11 depicts in schematic section an alternative version of an articulating socket element, with associated screw-like part, which forms part of the thumb joint prosthesis.

The drawings depict a preferred version of a thumb joint prosthesis 1 which has a first prosthesis component 2 and a second prosthesis component 3. The first prosthesis component 2 has a first screw-like part 4 and the second prosthesis component 3 has a second screw-like part 5. The first prosthesis component 2 has in addition an articulating socket element 6 and the second prosthesis component 3 has in addition an articulating ball element 7. Within the scope of the invention, the first screw-like part and the socket element may be integral (see FIG. 11). Within the scope of the invention, the second screw-like part and the ball element may also be integral. In the version according to FIGS. 2 and 3, however, the first and second screw-like parts 4, 5 each have a fastening portion 8, 9 respectively which in the version depicted each have a hole 10, 11 respectively for the socket element 6 and the ball element 7 respectively.

The hole 10 in the first screw-like part 4 extends, in the form of a hollow at a first end edge 12 at which said part 4 has its largest diameter, in an axial direction towards a second end edge 13 of said part 4 at which the latter has its smallest diameter. The hole 10 is preferably centred with a geometrical centreline CL1 extending in the axial direction of the part 4.

The hole 11 in the second screw-like part 5 extends, in the form of a hollow at a first end edge 14 at which said part 5 has its largest diameter, in an axial direction towards a second end edge 15 of said part 5 at which the latter has its smallest diameter. The hole 11 is centred with a geometrical centreline CL2 extending in the axial direction of the part 5.

The hole 10 in the first screw-like part 4 has its largest diameter at the first end edge 12 and its sidewalls 16 are of conical shape, their smallest diameter being at the bottom 17 of the hole 10, i.e. the shape of the hole 10 narrows conically towards its bottom 17.

The hole 11 in the second screw-like part 5 has its largest diameter at the first end edge 14 and its sidewalls 18 are of conical shape, their smallest diameter being at the bottom 19 of the hole 11, i.e. the shape of the hole 11 narrows conically towards its bottom 19.

At least the prosthesis component 2 with the socket element 6 has according to the invention an abutment portion 20 for abutment against the bone to which the screw-like part of this prosthesis component, said first screw-like part 4, is intended to be screwed firmly, which in the version depicted is the trapezium. The abutment portion 20 affords a large contact surface against the bone and prevents the prosthesis component 2, or more particularly the latter's screw-like part 4, since the abutment portion is with advantage configured thereon, from sinking into the bone. Theoretically, such an abutment portion might also be disposed on the prosthesis component 3 with the ball element 7, i.e. with advantage on the screw-like part 5, but the positive effect thereof would not be so marked as on the first screw-like part, particularly as this prosthesis component is primarily intended, as in the version depicted, for the first metacarpal bone, in which case the second screw-like part may be made longer for optimum primary fixation.

The socket element 6 has a socket 21 which forms a concave articulating surface 22. A fastening spigot 23 protrudes in an axial direction from the outside of the socket 21. The fastening spigot 23 has an axial outside 25 which narrows conically towards its end edge 24. The shape and size of the fastening spigot 23 and the shape and size of the hole 10 in the first screw-like part 4 are chosen such that they can by being pressed together in an axial direction constitute a push fit, i.e. a connection such as to make it possible for the socket element 6 and the first screw-like part 4 to be caused, by being pressed together, to remain firmly in position adjacent to one another.

The abutment portion 20 on the first screw-like part 4 in the preferred version depicted is disposed in substantially the same plane as the end edge 12 with the hole 10 in said screw-like part for the fastening spigot 23 of the socket element 6. The abutment portion takes the form in the preferred version depicted of a collar 20 running round the first screw-like part 4 in substantially the same plane as the end edge 12 with the hole 10 for the fastening spigot 23 of the socket element 6. The underside of the collar 20 may be caused to abut against the outside of the trapezium or alternatively a recess 43 may be formed in the trapezium for the collar so that the collar does not protrude from the bone. The abutment is effected against, or alternatively the recess 43 is formed in, the surface of the trapezium into which the first screw-like part 4 is screwed.

The collar 20 may where necessary be provided with holes 26 running through it for fastening means, preferably screws, in order to strengthen the primary fixation of the first screw-like part 4 in the trapezium.

Optimum stability of fixation of the first screw-like part 4 in the version depicted is achieved primarily by said part having a length which substantially corresponds to the length of the trapezium minus about 2.5 mm. The length of the first screw-like part 4 varies depending on the anatomy of the patient being fitted with the prosthesis. Suitable lengths appropriate in most cases have been found to be 7, 8.5 and 10 mm. The end edge 13 of the first screw-like part 4 is with advantage planar for its optimum abutment against the bone/cartilage boundary on the trapezium.

The ball element 7 has a substantially spherical ball 27 which constitutes a convex articulating surface 28 of such a shape that it fits into the surface 22 of the socket 21 so that said surfaces 22, 28 can slide against one another and make it possible for the joint to function. A fastening spigot 29 protrudes in an axial direction from the ball 27 and has an outside 31 which narrows conically in the axial direction towards its end edge 30. The length of the portion between the ball 27 and the fastening spigot 29 may vary depending on the anatomy of the patient being fitted with the prosthesis. The shape and size of the fastening spigot 29 and the shape and size of the hole 11 in the second screw-like part 5 are chosen such that they can, by being pressed together in an axial direction, constitute a push fit, i.e. a connection such as to allow the ball element 7 and the second screw-like part 5 to be caused, by being pushed together, to remain firmly in position adjacent to one another.

The first screw-like part 4 has at least one recess 32 configured to allow a shank 33 of a screwing tool 34, e.g. a screwdriver, to be inserted in the recess 32 in order to screw the first screw-like part 4 firmly to the respective bone, which in the version depicted is the trapezium. The second screw-like part 5 has at least one recess 35 configured to allow a shank 33 of a screwing tool 34 to be inserted in the recess 35 in order to screw the second screw-like part 5 firmly to the respective bone, which in the version depicted is the first metacarpal bone.

Each recess 32, 35 is disposed within the respective hole 10, 11 in the first and second screw-like parts 4, 5 respectively. The shank 33 of the screwing tool 34 is so configured that it can be inserted as far as, and into engagement with, the respective recess 32, 35 by being passed through the hole 10, 11 without damaging the sidewalls 16, 18 of these holes 10, 11. Each recess 32, 35 is preferably disposed in the bottom 17, 19 of the respective hole 10, 11.

Like the hole 10, 11, each recess 32, 35 is with advantage centred with the respective centreline CL1, CL2. Each recess 32, 35 may also be a non-circular recess, i.e. a polygonal recess, e.g. a hexagonal recess or the like. The shank 33 of the screwing tool 34 will of course be appropriate to the shape of the recess 32, 35 so that the respective screw-like part 4, 5 can be turned by turning the screwing tool 34 in order to screw each of these parts into the respective bone.

With advantage, the outside of the respective first and second screw-like parts 4, 5 narrows conically from the first end edge 12, 14 towards the second end edge 13, 15. The conical shape extends preferably, but not necessarily, all the way between said first end edges 12, 14 and second end edges 13, 15 respectively. Each of the first and second screw-like parts 4, 5 does of course have outer threads 36, 37 which preferably have self-tapping characteristics.

As depicted in FIG. 2, the outer threads 36 on the first screw-like part 4 may begin at the latter's first end edge 12 and end at its second end edge 13 so that the first screw-like part has a threaded end.

Correspondingly, as depicted in FIG. 3, the outer threads 37 on the second screw-like part 5 may begin at the latter's first end edge 14 and end just before its second end edge 15 so that the second screw-like part has an unthreaded leading end.

Each of the first and second screw-like parts 4, 5 may also be unthreaded on elongate portions 4a, 5a which extend in an axial direction along the respective first and second screw-like parts 4, 5 in order to divide the outer threads 36, 37 into two or more, e.g. four, threaded sections.

As previously mentioned, the thumb joint prosthesis 1 described above is disposed in the trapezium and the first metacarpal bone respectively. These bones are represented schematically in FIG. 1 by solid lines and are designated 38 and 39 respectively. As the first screw-like part 4 in a preferred version is screwed into the trapezium 38, this part is configured thicker and shorter than the second screw-like part 5 and with a larger hole 10 than the hole 11 in the second screw-like part 5. As previously indicated, the second screw-like part 5 is screwed firmly to the first metacarpal bone 39. Like the first screw-like part 4, the second screw-like part 5 is also available in various different lengths, e.g. lengths of 20, 24, 28, 32 and 36 mm.

FIGS. 6-10 depict an example of how the first and second screw-like parts 4, 5 can be applied in the bones 38, 39 respectively.

As depicted in FIG. 6, a straight incision about 3-5 cm long and centred across the thumb joint is effected to allow access to the thumb joint. The proximal end of the first metacarpal bone 39 is exposed and about 3 mm of it is re-sectioned perpendicular to longitudinal direction of the bone. A solid conical drill 40 with a measuring scale is used to drill a duct into the bone 39. The drilling is with advantage performed manually and monitored by radiography. The length of the second screw-like part 5 is read off at the re-sectioned surface of the bone 39 when the drill meets resistance from cortical bone. The length read off on the second screw-like part 5 is adopted and this part is screwed in (FIG. 7) so that its proximal portion is in the same plane as the re-sectioned surface on the bone 39. The fixation is checked. If the fixation is insufficient, it is recommended to change to a screw-like part 5 of the same length but larger size.

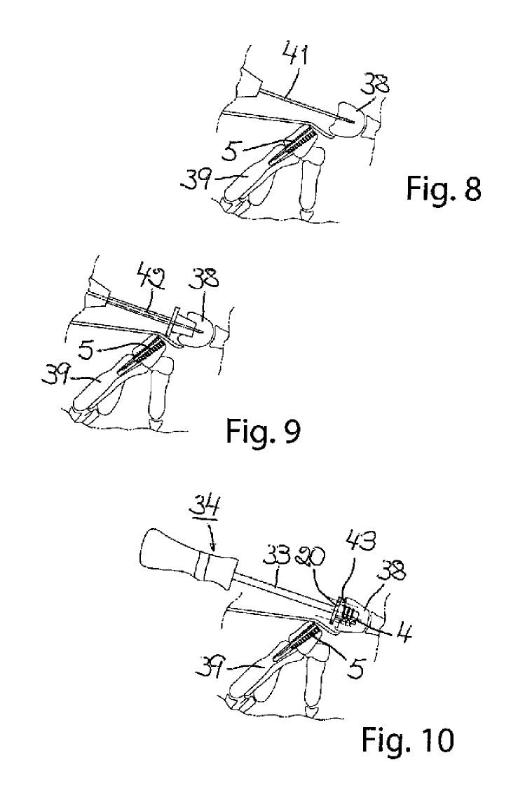

As depicted in FIG. 8, a hole in the trapezium 38 is centred and thereafter drilled with a guide pin until the tip of the pin reaches the subchondral bone/cartilage boundary. The drilling is done under radiography. The required length of the first screw-like part 4 is read off on the pin 41. If the length is between two available sizes, it is recommended to use the shorter. Thereafter a drill 42 is used to drill a hole of similar length in the trapezium 38, while at the same time a recess 43 for the collar 20 on the first screw-like part 4 is preferably cut into the bone (FIG. 9). This may be done manually or by machine. The pin 41 and the drill 42 are removed. After flushing clean, the first screw-like part 4 is screwed into the trapezium 38, advantageously by means of a screwdriver 34, until the collar 20 is in the same plane as the preferably re-sectioned surface of the trapezium 38 or in the recess 43 therein (FIG. 10). The fact that the first screw-like part 4 is slightly conical makes immediate success possible.

Where necessary, angle-stable screws (not depicted) are inserted in the collar 20 on the first screw-like part 4. Drilling guides (not depicted) are screwed initially into holes 26 in the collar 20.

This is followed by drilling and checking by radiography that the screws do not go beyond the trapezium 38 or into any adjacent joint. The length of the screws is read off on the drill (not depicted). The drill guides and the drill are removed and screws of the correct length are screwed into at least two of the holes 26, preferably three holes. During the screwing in, make sure also that no screw interferes with the socket element 6 in the trapezium 38.

Finally, the socket element 6 is put in place on the first screw-like part 4. A trial prosthesis (ball) is used to test that the thumb joint is fully movable. Subluxation of the thumb should be possible, i.e. pulling the thumb in its longitudinal direction so that the ball lifts somewhat from the socket element 6. The correct size of ball element 7 is chosen and is put in place on the second screw-like part 5. The incision is sutured.

The respective first and second screw-like parts 4, 5 may be made of at least one material, while the socket element 6 and the ball element 7 may be made of at least one other material. Thus each of the first and second screw-like parts 4, 5 may have a core of metal material with an external layer of a material which is dissolvable when the first and second screw-like parts 4, 5 are implanted and which allows secondary fixation of the screw-like parts.

Said core may with advantage be made of a titanium alloy, while the dissolvable material may be or contain calcium phosphate, so that the abutment portion, preferably the collar 20, can “grow firmly” to the bone against which it abuts.

Thus the abutment portion, preferably the collar 20, on the first screw-like part 4 is provided on at the least the underside with a dissolvable material, preferably calcium phosphate.

Each of the first and second screw-like parts 4, 5 may alternatively be made entirely of a metal material, e.g. a titanium alloy.

The socket element 6 and the ball element 7 may be made of a cobalt-chromium alloy.

If the mutually cooperating articulating or sliding surfaces 22, 28 on the various prosthesis components 2, 3 are made of metal, this will help to prevent the occurrence of osteolysis.

As well as the thumb joint prosthesis described above being simple and easy to apply, the surgical operation can be minor and only involve cutting small portions of the respective bones 38, 39 in order to fasten the screw-like parts 4, 5.

As previously mentioned, it is possible within the scope of the present invention for the prosthesis component 2 with the socket element 6 and associated screw-like part 4 to be so configured that the socket element and the screw-like part are integral with one another. In that case, the socket element is with advantage so configured that the socket 21 is substantially directly adjacent to the screw-like part, which may instead of threads have grooves or recesses 44 to facilitate the fixation of the prosthesis component 2 with bone cement (see FIG. 11).

In that case, the abutment portion 20 is configured in substantially the same plane as the end edge with the socket 21 in the prosthesis component, i.e. in the first screw-like part 4 of the prosthesis component. The abutment portion 20 may have holes 45 through which bone cement can be pushed up when the prosthesis component 2 is being applied to the respective bone, i.e. primarily the trapezium 38. The fixation of the prosthesis component 2 is thereby further improved.

The abutment portion 20 may if so desired be caused to have a shape substantially corresponding to the shape of the bone to which the prosthesis component 2 is applied, i.e. primarily the trapezium 38.

The invention is not limited to the version described above but may be varied within the scope of the claims set out below. Thus the configuration of the prosthesis components 2, 3 may vary. Likewise, the holes 10, 11 in the screw-like parts 4, 5 may be partly conical or may also, where appropriate, be of some other shape than conical, and the socket element 6 and the ball element 7 may of course be adapted accordingly. The abutment portion 20 may be of some other configuration than the circular collar shape depicted, and its thickness may vary, as also the number and shape of any holes 26 in it for fastening means. The push fit described may instead be some other type of connection device, and the recesses 32, 35 may be of some other advantageous shape than that described and be positioned in some other advantageous manner than that described. The socket and the ball of the socket element 6 and the ball element 7 respectively may be of some other shape than that depicted and the end edges 13, 15 of the respective first and second screw-like parts 4, 5 may have rounded corners 13a, 15a. Finally it may be noted that the screwing tool 34 may be some other suitable type of screwing tool than a screwdriver.

1. A joint prosthesis comprising two prosthesis components (2, 3) intended to be applied respectively to the trapezium (38) and the first metacarpal bone (39) of a thumb joint,
each prosthesis component (2, 3) having a first or second screw-like part (4 or 5) intended to be fastened, e.g. screwed firmly, to one of said bones (38, 39) or alternatively to be fastened thereto by means of bone cement, and
one prosthesis component (2) having an articulating socket element (6) and the other prosthesis component (3) an articulating ball element (7),
characterised
in that at least the prosthesis component (2) with the socket element (6) has an abutment portion (20) for abutment against the bone (38) to which the screw-like part (4) of the prosthesis component is intended to be firmly screwed or be fastened by means of bone cement.

2. A joint prosthesis according to claim 1, characterised in that the abutment portion (20) is configured on the screw-like part (4) of said prosthesis (2).

3. A joint prosthesis according to claim 2, characterised in that the abutment portion (20) is configured on the screw-like part (4) of said prosthesis (2) so that it abuts against the outside of, or alternatively against one or more sides in a recess (43) in, the bone to which the screw-like part is screwed firmly or is fastened by means of bone cement.

4. A joint prosthesis according to claim 2 or 3,
in which the socket element (6) has a fastening spigot (23) which can be inserted in a hole (10) in the first screw-like part (4) in order to apply the socket element thereto, and
in which the ball element (7) has a fastening spigot (29) which can be inserted in a hole (11) in the second screw-like part (5) in order to apply the ball element thereto,
characterised
in that the abutment portion (20) is disposed in substantially the same plane as the end edge (12) with the hole (10) in the first screw-like part (4) for the fastening spigot (23) of the socket element (6).

5. A joint prosthesis according to claim 4, characterised in that the abutment portion takes the form of a collar (20) which runs round the first screw-like part (4) in substantially the same plane as the end edge (12) with the hole (10) for the fastening spigot (23) of the socket element (6).

6. A joint prosthesis according to claim 5, characterised in that the collar (20) has holes (26) for fastening means for screwing the first screw-like part (4) firmly to the bone (38).

7. A joint prosthesis according to any one of the above claims,
in which the prosthesis (2) with the socket element (6) is intended to be screwed firmly into the trapezium (38),
characterised
in that the screw-like part (4) of said prosthesis (2) has a length substantially corresponding to the length of the trapezium (38) minus about 2.5 mm.

8. A joint prosthesis according to claim 7, characterised in that the screw-like part (4) for the prosthesis component (2) with the socket element (6) has a length of 7 or 8.5 or 10 mm.

9. A joint prosthesis according to any one of claims 4-8
characterised
in that the first and second screw-like parts (4, 5) are each provided with at least one recess (32, 35) configured to allow a shank (33) of a screwing tool (34) to be inserted therein in order to screw the first screw-like part (4) and the second screw-like part (5) firmly to the respective bone (38, 39),
that the recess (32, 35) is disposed in the bottom (17, 19) of the hole (10, 11) in the respective screw-like part (4, 5), and
that the shank (33) can be inserted in said recess (32, 35) by being passed through the hole (10, 11) in the first screw-like part (4) and the second screw-like part (5) respectively.

10. A joint prosthesis according to claim 9, characterised in that the recess (32, 35) is a non-circular recess.

11. A joint prosthesis according to claim 10, characterised in that the recess (32, 35) is a polygonal recess.

12. A joint prosthesis according to claim 11, characterised in that the recess (32, 35) is a hexagonal recess.

13. A joint prosthesis according to any one of claims 9-12, characterised in that the recess (32, 35) and the hole (10, 11) are centred or substantially centred with a common geometric centreline (CL1, CL2) which extends through the first screw-like part (4) and the second screw-like part (5) respectively.

14. A joint prosthesis according to any one of claims 4-13, characterised in that the hole (10, 11) in the respective first and second screw-like parts (4, 5) and the fastening spigots (23, 29) of the socket element (6) and the ball element (7) respectively is so configured that the first and second screw-like parts and the socket element respectively and the ball element respectively are adapted to constituting push fits which make it possible for the first screw-like part (4) and the socket element (6) to be caused, by being pressed together, to remain firmly in position adjacent to one another and for the second screw-like part (5) and the ball element (7) to be caused, by being pressed together, to remain firmly in position adjacent to one another.

15. A joint prosthesis according to claim 14, characterised in that the hole (10, 11) in the respective first and second screw-like parts (4, 5) and the fastening spigots (23, 29) of the socket element (6) and the ball element (7) respectively is conical.

16. A joint prosthesis according to any one of the above claims, characterised in that the respective first and second screw-like parts (4, 5) are of externally conical shape and have external threads (36, 37).

17. A joint prosthesis according to claim 16, characterised in that the external threads (36) of the first screw-like part (4) begin at a first end edge (12) of the latter and end at a second end edge (13) of it so that the first screw-like part has a threaded end.

18. A joint prosthesis according to claim 16 or 17, characterised in that the external threads (37) of the second screw-like part (5) begin at a first end edge (14) of the latter and end at a second end edge (15) of it so that the second screw-like part has an unthreaded leading end.

19. A joint prosthesis according to any one of claims 16-18, characterised in that the external threads (36, 37) of the respective and second screw-like parts (4, 5) have self-tapping characteristics.

20. A joint prosthesis according to any one of claims 16-19, characterised in that the respective first and second screw-like parts (4, 5) are unthreaded on elongate portions (4a, 5a) of them which extend in an axial direction along the first and second screw-like parts (4, 5) respectively in order to divide the external threads (36, 37) into a number of threaded sections.

21. A joint prosthesis according to any one of the above claims, characterised in that the ball (27) of the ball element (7) is spherical and that the socket element (6) has a corresponding socket (21).

22. A joint prosthesis according to any one of the above claims, characterised in that the first screw-like part (4) and the second screw-like part (5) are each made of at least one material and that the socket element (6) and the ball element (7) are each made of at least one other material.

23. A joint prosthesis according to any one of the above claims, characterised in that the first screw-like part (4) and the second screw-like part (5) each have a core of metal material and at least one external layer of material which is dissolved when the respective first and second screw-like parts are implanted.

24. A joint prosthesis according to claim 23, characterised in that the metal material of the core is a titanium alloy and that the dissolvable material is or comprises calcium phosphate.

25. A joint prosthesis according to claim 23 or 24, characterised in that the abutment portion (20) on the first screw-like part (4) is provided on at least the underside with a dissolvable material, preferably calcium phosphate.

26. A joint prosthesis according to any one of claims 1-22, characterised in that the first screw-like part (4) and the second screw-like part (5) are each made of a metal material, e.g. a titanium alloy.

27. A joint prosthesis according to any one of the above claims, characterised in that the socket element (6) and the ball element (7) are each made of a cobalt-chromium alloy.

28. A joint prosthesis according to any one of claims 4-27, characterised in that the first screw-like part (4) is thicker and shorter and has a larger hole (10) than the second screw-like part (5).

29. A joint prosthesis which has two prosthesis components (2, 3) intended to be applied respectively to the trapezium (38) and the first metacarpal bone (39) of a thumb joint,
in which each prosthesis component (2, 3) has a first or second screw-like part (4, 5) respectively intended to be screwed firmly to one of said bones (38, 39),
in which one prosthesis component (2) has an articulating socket element (6) and the other prosthesis component (3) an articulating ball element (7), and
in which the prosthesis component (2) with the socket element (6) is intended to be screwed firmly into the trapezium (38),
characterised
in that the first screw-like part (4) for the prosthesis component (2) with the socket element (6) has a length substantially corresponding to the length of the trapezium (38) minus about 2.5 mm.

30. A joint prosthesis according to claim 29, characterised in that the first screw-like part (4) for the prosthesis component (2) with the socket element (6) has a length of 7 or 8.5 or 10 mm.

31. A joint prosthesis according to any one of the above claims, characterised in that the abutment portion (20) has a shape substantially corresponding to the shape of the articulating surface on the bone (38) to which the screw-like part (4) of the prosthesis component (2) is intended to be firmly screwed.

32. A joint prosthesis according to any one of claims 1-3,
characterised
in that the socket element (6) and associated screw-like part (4) for said prosthesis component (2) are integral with one another, and
that the abutment portion (20) is configured in substantially the same plane as the end edge with the socket (21) of the socket element (6) in the prosthesis component (2).

33. A joint prosthesis according to claim 32,
in which the prosthesis component (2) with the socket element (6) is intended to be fastened to one of said bones (38) by means of bone cement,
characterised
in that the abutment portion (20) has holes or perforations (45) for bone cement.

34. A joint prosthesis according to claim 32 or 33, characterised in that the abutment portion (20) has a shape substantially corresponding to the shape of the articulating surface on the bone (38) to which the screw-like part (4) of the prosthesis component (2) is intended to be fastened by means of bone cement.

35. A joint prosthesis according to any one of claims 32-34, characterised in that the socket element (6) and the ball element (7) are made of metal material.
